(19) United States
(12) Patent Application Publication
(10) Pub. No.: US 2010/0150841 A1
(43) Pub. Date: Jun. 17, 2010

(54) Use of Tumor Necrosis Factor-Alpha P75 Receptor for the Reduction of Inflammation

(75) Inventors: David A. Goukassian, West Newton, MA (US); Raj Kishore, Chicago, IL (US)

Correspondence Address: EDWARDS ANGELL PALMER & DODGE LLP, P.O. BOX 55874, BOSTON, MA, 02205, US

(73) Assignee: CARITAS ST. ELIZABETH MEDICAL CENTER OF BOSTON, IN, BOSTON, MA (US)

(21) Appl. No.: 12/513,017

(22) PCT Filed: Oct. 31, 2007
(86) PCT No.: PCT/US07/22977

§ 371 Date: Mar. 1, 2010

Related U.S. Application Data
(60) Provisional application No. 60/855,997, filed on Oct. 31, 2006.

(51) Int. Cl. A61K 49/00; A61K 31/7088; A61K 45/00; C12N 15/74; G01N 33/567; A61P 37/06

(52) U.S. Cl. 424/9.2; 514/44 R; 424/93.21; 435/320.1; 435/7.21

(57) ABSTRACT

As described below, the present invention features compositions and methods for increasing TNFR2/p75 receptor signaling to reduce inflammation. Methods for increasing TNFR2/p75 expression or signaling may also be used to increase the survival of resident cells or transplanted stem cells (e.g., bone marrow derived or peripheral blood derived stem cells) in a damaged tissue.

### CROSS-REFERENCE TO RELATED APPLICATION

This application claims the benefit of U.S. Provisional Application No. 60/855,997, filed on Oct. 31, 2006, the entire contents of which are incorporated herein by reference.

### BACKGROUND OF THE INVENTION

Aging is associated with an increased risk of atherosclerotic disease of the coronary and peripheral arteries. In either vascular system, the extent of ischemic damage and degree of subsequent functional recovery after arterial obliteration largely depends on the development of new collateral blood vessels. Aging is associated with impaired angiogenesis in murine and rabbit limb ischemia models. Aging is also accompanied by a steady decline in immune functions, such as defects in signaling pathways and altered expression of cytokines, such as interferon-gamma (IFNγ) and vascular endothelial growth factor (VEGF). Angiogenesis is associated with perivascular inflammation and monocyte/macrophage accumulation.

Tumor necrosis factor alpha (TNF-α), a macrophage/monocyte-derived pluripotent mediator, can function as an angiogenic factor in one system and as an anti-angiogenic factor in another. These mutually exclusive effects have been attributed to TNF-α concentration and duration of exposure; that is, low concentrations and short exposure is angiogenic, whereas high concentrations and prolonged exposure is anti-angiogenic. TNF-α has been reported to induce the expression of many important immune- and angiogenesis-related genes through two different TNF-α receptors: TNF-αR1 (p55) and TNF-αR2 (p75). In various vascular endothelial cells, TNF-α increased the expression of the well-known angiogenic factors VEGF, basic fibroblast growth factor (bFGF), and interleukin-6 (IL-6). The role of the two distinct TNF-α receptors in mediating these responses are still unclear.

Given that angiogenesis is impaired in elderly individuals, this dysfunction may be related to alterations in TNF-α receptor expression. Methods of enhancing blood flow in the elderly are required to treat or prevent ischemia.

### SUMMARY OF THE INVENTION

As described below, the present invention features methods for increasing TNFR2/p75 receptor expression or signaling to reduce inflammation in damaged tissue. Methods for increasing TNFR2/p75 expression or signaling may also be used to increase the survival of resident cells or transplanted stem cells (e.g., bone marrow derived or peripheral blood derived stem cells) in a tissue. In one embodiment, the invention provides compositions and methods for the prevention or treatment of inflammation related to ischemic damage.

In one aspect, the invention provides a method of treating or preventing inflammation in a subject, the method comprising, contacting a cell of the subject (e.g., a human or animal subject) with a p75/TNFR2 nucleic acid molecule encoding a p75/TNFR2 polypeptide or a fragment thereof; and expressing the p75/TNFR2 polypeptide in the cell, wherein the method treats or prevents inflammation in the subject.

In a related aspect, the invention provides a method of treating or preventing tissue damage associated with inflammation in a subject, the method involving contacting a cell of the subject with a p75/TNFR2 polypeptide or a fragment thereof, thereby treating or preventing tissue damage associated with inflammation in the subject.

In various embodiments, the tissue damage or inflammation is associated with ischemia.

In another related aspect, the invention provides a method for repairing or regenerating a damaged tissue in a subject, the method involving contacting a cell of the subject with a nucleic acid molecule encoding a p75/TNFR2 polypeptide or a fragment thereof; and expressing the p75/TNFR2 polypeptide in the cell, thereby repairing or regenerating the damaged tissue.

In another related aspect, the invention provides a method of treating tissue damage in a subject involving contacting an adult stem isolated from the subject with a nucleic acid molecule encoding a p75/TNFR2 polypeptide or a fragment thereof; and administering the cell to the subject, wherein the method enhances angiogenesis.

In yet another aspect, the invention provides a method of treating or preventing inflammation in a subject having or at risk of developing ischemia, the method involving contacting a cell of the subject with an agent that increases (e.g., by 5%, 10%, 25%, 50%, 75%, 100%, 200%, or more) p75/TNFR2 receptor signaling thereby treating or preventing inflammation.

In yet another aspect, the invention provides a method for increasing the survival of a transplanted stem cell in a damaged tissue, the method involving contacting a cell in a damaged tissue of the subject with a nucleic acid molecule encoding a p75/TNFR2 polypeptide or a fragment thereof; expressing the p75/TNFR2 polypeptide in the cell, and transplanting the cell into the damaged tissue, where the method increases the survival of the stem cell in the damaged tissue.

In yet another aspect, the invention provides a pharmaceutical composition labeled for the treatment of inflammation involving an effective amount of an expression vector encoding a human p75/TNFR2 polypeptide or a fragment thereof in a pharmaceutically acceptable excipient, wherein the p75/TNFR2 polypeptide is operably linked to a promoter sufficient to drive expression of the p75/TNFR2 polypeptide in a mammalian cell. In one embodiment, the vector is a viral vector. In another embodiment, the promoter is sufficient to drive expression in a peripheral blood- or bone marrow-derived endothelial progenitor cell or a bone marrow-derived stem cell.

In yet another aspect, the invention provides a kit for the treatment or prevention of inflammation, the kit containing an effective amount of an expression vector encoding a human p75/TNFR2 polypeptide or a fragment thereof in a pharmaceutically acceptable excipient, wherein the p75/TNFR2 polypeptide is operably linked to a promoter sufficient to drive expression of the p75/TNFR2 polypeptide in a mammalian cell, and written instructions for the use of the kit.

In yet another aspect, the invention provides a method of monitoring a subject being treated for inflammation, the method involving administering a treatment that enhances the expression of a p75/TNFR2 polypeptide in a cell of the subject; and measuring inflammation in a tissue of the subject relative to a reference, wherein an decrease in inflammation indicates a reduced severity of ischemia in the subject. In one embodiment, the reference is the level of inflammation previously present in the subject or in a biological sample derived from the subject at an earlier time point.

In yet another aspect, the invention provides a method for identifying a candidate compound useful for the treatment of inflammation, the method involving the steps of contacting a cell expressing p75/TNFR2 nucleic acid molecule with a candidate compound; and detecting an increase in p75/TNFR2 expression in the cell relative to a reference, wherein an increase in p75/TNFR2 expression identifies the candidate compound as a compound useful for the treatment of ischemia. In one embodiment, the method identifies a compound that increases transcription or translation of a p75/TNFR2 nucleic acid molecule, or increases stabilization and decreases degradation of gene product of p75/TNFR2 nucleic acid molecule.

In a related aspect, the invention provides a method for identifying a candidate compound useful for the treatment of inflammation, the method involving the steps of contacting a cell expressing p75/TNFR2 polypeptide with a candidate compound; and detecting an increase in the level of p75/TNFR2 polypeptide in the cell relative to a reference level, wherein an increase in the level of p75/TNFR2 polypeptide identifies a candidate compound useful for the treatment of ischemia.

In another related aspect, the invention provides a method for identifying a candidate compound useful for the treatment of ischemia, the method involving the steps of contacting a cell expressing a p75/TNFR2 polypeptide with a candidate compound; and detecting an increase in the biological activity of the p75/TNFR2 polypeptide in the cell contacted with the candidate compound with a reference level of biological activity wherein the candidate compound as a candidate compound that useful for the treatment of ischemia.

In various embodiments of any of the above-aspects, the cell is a skeletal muscle cell, endothelial cell, resident stem cell, satellite cell, cardiomyocyte, or cardiac stem cell. In various embodiments of any of the above-aspects, the method enhances the local release of angiogenic growth factors (e.g., vascular endothelial growth factor (VEGF), hepatocyte growth factor (HGF), basic fibroblast growth factor (bFGF), angiopoietin 1, angiopoietin 2 and monocyte chemotactic protein-1 (MCP-1)) and cytokines in the tissue. In various embodiments of any of the above aspects, the method further involves of administering to the subject an angiogenic factor or an endothelial cell mitogen (e.g., acidic and basic fibroblast growth factors, vascular endothelial growth factor, epidermal growth factor, transforming growth factor α and β, platelet-derived endothelial growth factor, platelet-derived growth factor, hepatocyte growth factor, insulin like growth factor, erythropoietin, colony stimulating factor, macrophage-CSF, granulocyte/macrophage CSF and nitric oxide synthase). In various embodiments of any of the above aspects, the cell in vivo or in vitro. In various embodiments of any of the above aspects, the method further comprises the step of delivering the cell (e.g., directly or systemically) to a subject. In still other embodiments of the above aspects, the nucleic acid molecule is present in a vector (e.g., a viral vector, such as a cell specific over expression viral or non-viral vector) and the nucleic acid molecule is positioned for expression. In various embodiments of any of the above aspects, the tissue damage is related to ischemia. In still other embodiments of any of the above aspects, the method treats an inflammatory disease that is arthritis, rheumatoid arthritis, Crohn's disease, ulcerative colitis, asthma, chronic obstructive pulmonary disease, polymylagia rheumatica, giant cell arteritis, systemic lupus erythematosus, atopic dermatitis, multiple sclerosis, myasthenia gravis, psoriasis, ankylosing spondylitis, or psoriatic arthritis; or treats a condition that is myocardial infarction, heart failure, congestive heart failure, a stroke, a transient ischemic episode, a reperfusion injury, physical injury, renal failure, a secondary exsanguination, stroke, and traumatic brain injury, a transient ischemic attack. In still other embodiments of the above aspects, the subject is identified as having a reduction in p75/TNFR2 expression relative to a reference (e.g., the level present in a young adult) or the subject is identified as elderly.

The invention provides compositions and methods for treating tissue damage featuring TNFR2/p75. Other features and advantages of the invention will be apparent from the detailed description, and from the claims.

### DEFINITIONS

### Definitions

By “p75/TNFR2 polypeptide” is meant a protein or fragment thereof having substantial identity to the amino acid sequence of p75/TNFR2 provided at GenBank Accession No. NP_{—}001057 that reduces inflammation, promotes angiogenesis or has TNF binding activity.

The amino acid sequence of human p75/TNFR2 (GenBank Accession No. NP_{—}001057) is provided below:

| | |
|---|---|
| 1 | mapvavwaal avglelwaaa halpaqvaft pyapepgstc rlreyydqta qmccskcspg |
| 61 | qhakvfctkt sdtvcdsced stytqlwnwv peclscgsrc ssdqvetqac treqnrictc |
| 121 | rpgwycalsk qegcrlcapl rkcrpgfgva rpgtetsdvv ckpcapgtfs nttsstdicr |
| 181 | phqicnvval pgnasmdavc tstsptrsma pgavhlpqpv strsqhtqpt pepstapsts |
| 241 | fllpmgpspp aegstgdfal pvglivgvta lglliigvvn cvimtqvkkk plclqreakv |
| 301 | phlpadkarg tqgpeqqhll itapssssss lessasaldr raptrnqpqa pgveasgage |
| 361 | arastgssds spgghgtqvn vtcivnvcss sdhssqcssq asstmgdtds spsespkdeq |
| 421 | vpfskeecaf rsqletpetl lgsteekplp lgvpdagmkp s |

By “p75/TNFR2 nucleic acid molecule” is meant a polynucleotide that encodes a p75/TNFR2 polypeptide. An exemplary p75/TNFR2 nucleic acid molecule is provided at NCBI Reference No. NM_{—}001066.

By “p75/TNFR2 biological activity” is meant TNF binding activity, inflammation reducing activity, or angiogenesis enhancing activity.

By “angiogenesis” is meant any alteration that benefits tissue perfusion. Angiogenesis includes the growth by sprouting of endothelial cells from existing blood vessels or the remodeling of existing vessels to alter size, maturity, direction or flow properties to improve blood perfusion of tissues. In one embodiment, angiogenesis increases the density of an existing vascular network. Angiogenesis is measured by any method known in the art, including by determining the number of capillaries per muscle fiber.

By “apoptosis” is meant the process of cell death wherein a dying cell displays a set of well-characterized biochemical hallmarks that include cell membrane blebbing, cell soma shrinkage, chromatin condensation, and DNA laddering. Cells that die by apoptosis include neurons (e.g., during the course of a stroke or ischemic injury) and myocytes, including cardiomyocytes, resident endothelial cells, cardiac stem cells, and sattelite cells (e.g., after myocardial infarction or a stroke or ischemic injury, or over the course of congestive heart failure).

By “effective amount” is meant the amount of a compound required to prevent, treat, or ameliorate the symptoms of a disease.

By “enhance” is meant increase. For example, an increase of at least 5%, 10%, 25%, 50%, 75% or 100% relative to a reference.

By “inflammation” is meant any excessive or undesirable immune response associated with tissue damage.

By “ischemia” is meant reduced blood flow to a tissue or organ relative to the level required for the maintenance of normal cell metabolism. Exemplary ischemic events include primary myocardial infarction, secondary myocardial infarction, angina pectoris (including both stable and unstable angina), congestive heart failure, sudden cardiac death, cerebral infarction, restenosis, syncope, ischemia, reperfusion injury, vascular occlusion, carotid obstructive disease, transient ischemic attack, and the like.

By “angiogenic factor” is meant any polypeptide or fragment thereof that enhances angiogenesis.

By “endothelial cell mitogen” is meant any polypeptide or fragment thereof that supports the proliferation of an endothelial cell.

By “ameliorate” is meant decrease, suppress, attenuate, diminish, arrest, or stabilize the development or progression of a disease.

By “disease” is meant any condition or disorder that damages or interferes with the normal function of a cell, tissue, or organ. Examples of diseases include bacterial invasion or colonization of a host cell.

An “expression vector” is a nucleic acid construct, generated recombinantly or synthetically, bearing a series of specified nucleic acid elements that enable transcription of a particular gene in a host cell. Typically, gene expression is placed under the control of certain regulatory elements, including constitutive or inducible promoters, tissue-preferred regulatory elements, and enhancers.

By “fragment” is meant a portion of a protein or nucleic acid that is substantially identical to a reference protein or nucleic acid. In some embodiments the portion retains at least 50%, 75%, or 80%, or more preferably 90%, 95%, or even 99% of the biological activity of the reference protein or nucleic acid described herein.

By “immunological assay” is meant an assay that relies on an immunological reaction, for example, antibody binding to an antigen. Examples of immunological assays include ELISAs, Western blots, immunoprecipitations, and other assays known to the skilled artisan.

By “isolated nucleic acid molecule” is meant a nucleic acid (e.g., a DNA) that is free of the genes which, in the naturally-occurring genome of the organism from which the nucleic acid molecule of the invention is derived, flank the gene.

By “operably linked” is meant that the polynucleotide of the invention (e.g., a DNA molecule) is positioned adjacent to a DNA sequence that directs transcription and translation of the sequence (i.e., facilitates the production of, for example, a recombinant polypeptide of the invention, or an RNA molecule).

By “promoter” is meant a polynucleotide sufficient to direct transcription.

By “reduces” or “increases” is meant a negative or positive alteration, respectively, of at least 10%, 25%, 50%, 75%, or 100%.

By “reference” is meant a standard or control condition.\

By “regenerate” is meant capable of contributing at least one cell to the repair or de novo construction of a tissue or organ.

By “repair” is meant to ameliorate damage or disease in a tissue or organ.

By “satellite cell” is meant a muscle stem cell that functions in muscle growth and repair. Typically satellite cells are found in mature muscle between the basal lamina and sarcolemma. Satellite cells are able to differentiate and fuse to augment existing muscle fibres and to form new fibres. These cells are involved in the normal growth of muscle, as well as in muscle regeneration following injury or disease. Satellite cells are described, for example, by McCroskery et al., J. Cell Biol. 162(6):1135-47, 2003.

By “stem cell” is meant any multipotent cell.

By “subject” is meant a mammal, including, but not limited to, a human or non-human mammal, such as a bovine, equine, canine, ovine, or feline.

“Therapeutic compound” means a substance that has the potential of affecting the function of an organism. A therapeutic compound may decrease, suppress, attenuate, diminish, arrest, or stabilize the development or progression of disease or disorder an organism.

By “vector” is meant a DNA molecule, usually derived from a plasmid or bacteriophage, into which fragments of DNA may be inserted or cloned. A recombinant vector will contain one or more unique restriction sites, and may be capable of autonomous replication in a defined host or vehicle organism such that the cloned sequence is reproducible. A vector contains a promoter operably linked to a gene or coding region such that, upon transfection into a recipient cell, an RNA is expressed. Preferably, the promoter drives expression in an endothelial progenitor cell, in a bone marrow derived stem or progenitor cell, or in resident tissue adult stem cells. Accordingly, the invention provides for the expression of a p75TNFR in a host cell, such as a resident stem cell, an endothelial cell, or an adult stem cell.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. C. in 500 mM NaCl, 50 mM trisodium citrate, 1% SDS, 35% formamide, and 100 .mu.g/ml denatured salmon sperm DNA (ssDNA). In a most preferred embodiment, hybridization will occur at 42° C. C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42.degree. C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

### BRIEF DESCRIPTION OF THE DRAWINGS

FIG. 1 includes four micrographs showing that bone marrow-derived wild-type (WT) and TNFR2/p75 knockout (p75KO) cells from young mice proliferate in old p75KO ischemic tissue (red and green double positive cells—arrows). Resident cells proliferate only in old p75KO mice that were transplanted with young wild-type marrow (pink cells—indicated by circles).

FIG. 2 includes four micrographs showing that bone marrow-derived WT cells from young mice survive in old p75KO ischemic tissue. No cells from young bone marrow-derived p75KO cells survive in old p75KO ischemic tissue.

### DETAILED DESCRIPTION OF THE INVENTION

The invention features compositions and methods that are useful for restoration or up-regulation of TNFR2/p75. Such methods improve recovery in damaged tissue through inhibition of apoptosis (cell death), inhibition of prolonged inflammation, and promotion of angiogenesis thereby increasing survival of the resident cells and adult stem cells in the hostile environment and creating a “welcome” tissue environment for other endogenous (bone marrow or peripheral blood-derived) stem/progenitor cells.

The invention is based, at least in part, on the discovery that there are decreases in expression of TNFR2/p75 in adult tissue and increases in TNF alpha in the surrounding tissue, especially in damaged tissue. Such increases result, for example, from ischemic damage, chronic inflammation, trauma, or genetic disease. Through restoration of the expression of TNFR2/p75 one can reduce tissue inflammation, thereby preventing the harmful effects of inflammation on transplanted and/or resident stem cells. In addition, restoration or up-regulation of decreased TNFR2/p75 in the adult tissue should improve survival and proliferation of transplanted stem/progenitor cells and activation, proliferation and differentiation of resident stem cells.

p75/TNFR2

The p75 receptor/TNFR2 is a 415-amino acid polypeptide with a single membrane-spanning domain and has an extracellular domain with sequence similarity to nerve growth factor receptor (Schall et al., “Molecular cloning and expression of a receptor for human tumor necrosis factor,” Cell 61: 361-370, 1990). Human p75/TNFR2 gene spans nearly 43 kb and consists of 10 exons and 9 introns (Beltinger et al., Physical mapping and genomic structure of the human TNFR2 gene. Genomics 35: 94-100, 1996). The amino acid sequence of p75/TNFR2 is provided at GenBank Accession No. NP_{—}001057. A nucleic acid sequence encoding the p75/TNFR2 polypeptide is provided at NM_{—}001066.

### P75/TNFR2 Polypeptides and Analogs

Overexpression of a P75/TNFR2 polypeptide or fragment reduces tissue inflammation, thereby preventing the harmful effects of inflammation and improving the survival and/or proliferation of transplanted stem/progenitor cells and activation, proliferation and differentiation of resident stem cells. Accordingly, methods of the invention are useful for the treatment of ischemia. Included in the invention are p75/TNFR2 polypeptides, analogs, or fragments thereof, that are modified in ways that enhance their ability to promote stem cell survival or reduce inflammation. In one embodiment, the invention provides methods for optimizing a p75/TNFR2 amino acid sequence or nucleic acid sequence by producing an alteration in the sequence. Such alterations may include certain mutations, deletions, insertions, or post-translational modifications. The invention further includes analogs of any naturally-occurring polypeptide of the invention. Analogs can differ from a naturally-occurring polypeptide of the invention by amino acid sequence differences, by post-translational modifications, or by both. Analogs of the invention will generally exhibit at least 85%, more preferably 90%, and most preferably 95% or even 99% identity with all or part of a naturally-occurring amino, acid sequence of the invention. The length of sequence comparison is at least 5, 10, 15 or 20 amino acid residues, preferably at least 25, 50, or 75 amino acid residues, and more preferably more than 100 amino acid residues. Again, in an exemplary approach to determining the degree of identity, a BLAST program may be used, with a probability score between e⁻³ and e⁻¹⁰⁰ indicating a closely related sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides of the invention by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., .beta. or .gamma. amino acids.

If desired, the polypeptide can be fused to a protein transduction domain that provides for transport across the cell membrane. Accordingly, polypeptides of the invention can be provided directly to a cell, tissue, or organ where a reduction in inflammation, stem cell activation, or tissue repair is required.

In addition to full-length polypeptides, the invention also includes fragments of any one of the polypeptides of the invention. As used herein, the term “a fragment” means at least 10, 25, 50, 75, 100, 150, or 200 amino acids. In other embodiments a fragment is at least 20 contiguous amino acids, at least 30 contiguous amino acids, or at least 50 contiguous amino acids, and in other embodiments at least 60 to 80 or more contiguous amino acids. Fragments of the invention can be generated by methods known to those skilled in the art or may result from normal protein processing (e.g., removal of amino acids from the nascent polypeptide that are not required for biological activity or removal of amino acids by alternative mRNA splicing or alternative protein processing events).

Non-protein p75/TNFR2 analogs having a chemical structure designed to mimic p75/TNFR2 functional activity can be administered according to methods of the invention. p75/TNFR2 analogs may exceed the physiological activity of the original polypeptide. Methods of analog design are well known in the art, and synthesis of analogs can be carried out according to such methods by modifying the chemical structures such that the resultant analogs exhibit the angiogenesis promoting activity of a reference p75/TNFR2 polypeptide. These chemical modifications include, but are not limited to, substituting alternative R groups and varying the degree of saturation at specific carbon atoms of a reference p75/TNFR2 polypeptide. Preferably, the p75/TNFR2 analogs are relatively resistant to in vivo degradation, resulting in a more prolonged therapeutic effect upon administration. Assays for measuring functional activity include, but are not limited to, those described in the Examples below.

### Treatment of Tissue Damage

The increased expression of p75/TNFR2 in a cell prevents or treats inflammation and promotes muscle regeneration. Accordingly, the invention provides for the treatment of a variety of diseases and disorders associated with inflammatory processes. For example, the application prevents or treats inflammation associated with chronic inflammatory processes in adults having decreased levels of p75/TNFR2. Inflammatory diseases amenable to treatment by overexpression of p75/TNFR2 include but are not limited to arthritis, rheumatoid arthritis, Crohn's disease, ulcerative colitis, asthma, chronic obstructive pulmonary disease, polymylagia rheumatica, giant cell arteritis, systemic lupus erythematosus, atopic dermatitis, multiple sclerosis, myasthenia gravis, psoriasis, ankylosing spondylitis, or psoriatic arthritis.

Ischemia results when blood flow to a cell, tissue, or organ is interrupted. Tissue damage related to apoptotic cell death often results. Ischemic diseases are characterized by cell or tissue damage related to hypoxia. Exemplary ischemic diseases include, but are not limited to, ischemic injuries caused by a myocardial infarction, heart failure, congestive heart failure, a stroke, a transient ischemic episode, a reperfusion injury, physical injury, renal failure, a secondary exsanguination, or blood flow interruption resulting from any other primary diseases. The effects of ischemia are particularly devastating in the brain, when stroke, traumatic brain injury, myocardial infarction, or a transient ischemic attack limits blood flow to the tissues of the CNS. Accordingly, the invention provides therapeutic and prophylactic compositions useful for the treatment of ischemia that affects a variety of cells, tissues, or organs. The invention features compositions and methods that are useful for reducing inflammation associated with tissue damage and promoting collateral vessel development in an ischemic tissue. The invention generally features method of reducing apoptosis and inflammation in a subject having or at risk of developing ischemia. The method involves contacting a cell of the subject with a nucleic acid molecule encoding a p75 TNFR2 receptor polypeptide or a fragment thereof; and expressing the p75 TNFR2 receptor polypeptide in the cell, thereby reducing reducing inflammation and/or apoptosis in the ischemic tissue.

The present invention provides methods of treating disease and/or disorders or symptoms thereof related to inflammation or ischemia related tissue damage that comprise administering a therapeutically effective amount of a pharmaceutical composition comprising an agent that increases TNFR2/p75 receptor signalling, expression, or activity to a subject (e.g., a mammal such as a human). Thus, one embodiment is a method of treating a subject suffering from or susceptible to an ischemia-related disease or disorder or symptom thereof. The method includes the step of administering to the mammal a therapeutic amount of an amount of a compound herein sufficient to treat the disease or disorder or symptom thereof, under conditions such that the disease or disorder is treated.

The methods herein include administering to the subject (including a subject identified as in need of such treatment) an effective amount of a compound described herein, or a composition described herein to produce such effect (e.g., a reduction in inflammation). Identifying a subject in need of such treatment can be in the judgment of a subject or a health care professional and can be subjective (e.g. opinion) or objective (e.g. measurable by a test or diagnostic method).

The therapeutic methods of the invention (which include prophylactic treatment) in general comprise administration of a therapeutically effective amount of the compounds (e.g., TNFR2/p75) herein, such as a compound of the formulae herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The compounds herein may be also used in the treatment of any other disorders in which tissue damage or ischemia may be implicated.

In one embodiment, the invention provides a method of monitoring treatment progress. The method includes the step of determining a level of diagnostic marker (Marker) (e.g., any target delineated herein modulated by a compound herein, a protein or indicator thereof, etc.) or diagnostic measurement (e.g., screen, assay) in a subject suffering from or susceptible to a disorder or symptoms thereof associated with tissue damage or inflammation, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

Therapies provided by the invention include polynucleotide therapies, polypeptide therapies, as well as the delivery of endothelial progenitor cells (EPCs) expressing a heterologous p75/TNFR2. If desired, such cells may be removed from the patient, transfected, and then returned to the patient for the treatment of ischemia.

Delivery of therapeutic agents to ischemic tissues (e.g., muscle tissue, including cardiac tissue, and neural tissue, including the CNS) can be achieved by several methods. One method relies on neurosurgical techniques. In the case of gravely ill patients, surgical intervention is warranted despite its attendant risks. For instance, therapeutic agents can be delivered by direct physical introduction into the CNS, such as intraventricular, intralesional, or intrathecal injection. Intraventricular injection can be facilitated by an intraventricular catheter, for example, attached to a reservoir, such as an Ommaya reservoir. Methods of introduction are also provided by rechargeable or biodegradable devices.

In addition, the invention provides methods of screening for compounds that increase the biological activity or expression of p75/TNFR2 or that inhibit the biological activity or expression of p75/TNFR2. Such compounds are useful for enhancing angiogenesis, limiting the tissue damage associated with ischemia (e.g., by enhancing the survival of cells at risk of cell death associated with ischemia). Compounds that enhance p75/TNFR2 biological activity (e.g., angiogenesis enhancing activity) or expression may be used to treat or prevent ischemia in cells, tissues, or organs. Individuals at increased risk of an ischemic disease due to a hereditary condition are also candidates for such treatment.

p75/TNFR2 Polynucleotide Therapy

Therapy featuring a nucleic acid molecule encoding a p75 receptor/TNFR2 polypeptide, variant, or fragment thereof is one therapeutic approach for treating inflammation, particularly inflammation associated with ischemia, and for promoting the repair or regeneration of a muscle damaged by ischemia. In one embodiment, the methods of the invention increase the proliferation or survival of transplanted or resident stem cells. Such nucleic acid molecules can be delivered to cells (e.g., endothelial progenitor cells, or bone marrow derived cells or tissue adult stem cells) of a subject before, during, or after an ischemic episode. Such delivery may take place in vivo or ex vivo. In one embodiment, a human EPC is removed from a donor, transfected with a polynucleotide ex vivo, and then injected into a recipient patient in need thereof. Polynucleotide therapy has been successfully used to enhance angiogenesis. For example, the promotion of angiogenesis in the treatment of ischemia was demonstrated in a rabbit model and in human clinical trials with VEGF using a Hydrogel-coated angioplasty balloon as the gene delivery system (Takeshita, et al., Laboratory Investigation, 75:487-502 (1996); Isner, et al., Lancet, 348:370 (1996)). Successful transfer and sustained expression of the VEGF gene in the vessel wall subsequently augmented neovascularization in the ischemic limb (Takeshita, et al., Laboratory Investigation, 75:487-502 (1996); Isner, et al., Lancet, 348:370 (1996)). In addition, it has been demonstrated that direct intramuscular injection of DNA encoding VEGF into ischemic tissue induces angiogenesis, providing the ischemic tissue with increased blood vessels (U.S. Ser. No. 08/545,998; Tsurumi et al., Circulation 94(12):3281-90, 1996). The nucleic acid molecules must be delivered to the cells of a subject in a form in which they can be taken up so that therapeutically effective levels of a p75/TNFR2 polypeptide or fragment thereof can be produced.

Transducing viral (e.g., retroviral, adenoviral, and adeno-associated viral) vectors can be used for somatic cell gene therapy, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, a polynucleotide encoding a p75/TNFR2 receptor polypeptide variant, or a fragment thereof, can be cloned into a retroviral vector and expression can be driven from its endogenous promoter, from the retroviral long terminal repeat, or from a promoter specific for a target cell type of interest. Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77 S-83S, 1995). Retroviral vectors are particularly well developed and have been used in clinical settings (Rosenberg et al., N. Engl. J. Med 323:370, 1990; Anderson et al., U.S. Pat. No. 5,399,346). In one embodiment, a viral vector is used to administer a p75/TNFR2 receptor nucleic acid molecule systemically.

Non-viral approaches can also be employed for the introduction of a therapeutic nucleic acid molecule to a cell of a subject requiring a reduction in inflammation, or a promotion of muscle repair or regeneration. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Preferably the nucleic acids are administered in combination with a liposome and protamine.

Gene transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell. Transplantation of genes into the affected tissues of a subject (e.g., tissues subject to ischemia or requiring enhanced vascularization) can also be accomplished by transferring a normal nucleic acid into a cultivatable cell type ex vivo (e.g., an autologous or heterologous primary cell or progeny thereof), after which the cell (e.g., a bone marrow-derived cell or EPC, or its descendants) are injected into a targeted tissue (e.g., an ischemic tissue).

cDNA expression for use in polynucleotide therapy methods can be directed from any suitable promoter (e.g., the human cytomegalovirus (CMV), simian virus 40 (SV40), or metallothionein promoters, and regulated by any appropriate mammalian regulatory element. For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Another therapeutic approach included in the invention involves administration of a recombinant therapeutic, such as a recombinant p75 receptor/TNFR2 polypeptide, variant, or fragment thereof, either directly to the site of a potential or actual disease-affected tissue or systemically (for example, by any conventional recombinant protein administration technique). The dosage of the administered polypeptide depends on a number of factors, including the size and health of the individual subject. For any particular subject, the specific dosage regimes should be adjusted over time according to the individual need and the professional judgment of the person administering or supervising the administration of the compositions.

### Stem Cell Therapy

Transplantation of stem cells into a tissue is useful for the repair or regeneration of a tissue. The present invention promotes the survival and/or proliferation of transplanted or resident stem cells by reducing inflammation within a target tissue or organ. In one embodiment, expressing p75TNFR2 in at least one cell of the tissue activates quiescent stem cells, such as dormant satellite cells, cardiac stem cells, or other stem cells resident in the tissue. Activation of such resident stem cells provides for the repair or regeneration of a damaged tissue. By “activation” is meant induction of cell growth or proliferation.

Alternatively, stem cells can be transplanted into a tissue to promote tissue repair or regeneration. Expression of a p75/TNFR2 receptor in a cell of the tissue or in the transplanted stem cell promotes the survival, proliferation, and incorporation of the cell into the damaged tissue, thereby providing for tissue repair and regeneration. Stem cells can be derived from virtually any source known in the art. For example, hematopoietic stem cell derived from peripheral blood can provide sustained hematopoietic recovery. (See, for example, Kessinger et al., Blood 77, 211 (1991); Sheridan et al., Lancet 339, 640 (1992); Shpall et al., J. Clin. Oncol. 12, 28 (1994). This observation is now being exploited clinically as an alternative to bone marrow transplantation. By using techniques similar to those employed for hematopoietic stem cells, endothelial progenitor cells can be isolated from circulating blood. Such cells, once isolated, can be expanded in vitro and engineered to express one or more heterologous nucleic acid molecules. The cells are then delivered back to the donor, or to another subject, to achieve a therapeutic result.

To obtain the endothelial progenitor cell from peripheral blood about 5 ml to about 500 ml of blood is taken from a donor. Preferably, about 50 ml to about 200 ml of blood is taken. Endothelial progenitor cells are expanded in vivo by administration of recruitment growth factors, e.g., GM-CSF and IL-3, to the donor prior to removing the progenitor cells. Methods for obtaining and using hematopoietic progenitor cells in autologous transplantation are disclosed in U.S. Pat. No. 5,199,942, the disclosure of which is incorporated by reference. Alternatively, the cells are expanded ex vivo using, for example, the method disclosed by U.S. Pat. No. 5,541,103. Endothelial progenitor cells may be obtained from human mononuclear cells obtained from peripheral blood or bone marrow of the subject before treatment. Such cells may also be obtained from heterologous or autologous umbilical cord blood. In particular, endothelial progenitor cells may be obtained from the leukocyte fraction of peripheral blood. Endothelial progenitor cells may be isolated using antibodies that recognize endothelial progenitor cell specific antigens on immature human hematopoietic progenitor cells. For example, CD34 is commonly shared by endothelial progenitor cells and hematopoietic stem cells. CD34 is expressed by all hematopoietic stem cells but is lost by hematopoietic cells as they differentiate. Flk-1, a receptor for vascular endothelial growth factor (VEGF) is also expressed by both early hematopoietic stem cells and endothelial cells, but ceases to be expressed in the course of hematopoietic differentiation.

In vitro, endothelial progenitor cells differentiate into endothelial cells. Indeed, one can use a multipotentiate undifferentiated cell as long as it is still capable of becoming an endothelial cell in the presence of agents that promote its differentiation. In vivo, heterologous, homologous, and autologous endothelial cell progenitor grafts incorporate into sites of active angiogenesis or blood vessel injury by selectively migrating to such locations. Angiogenesis can be promoted in a subject by administering a potent angiogenesis factor, such as VEGF, alone or in combination with endothelial progenitor cells. Once the progenitor cells are obtained by a particular separation technique, they may be administered to a selected subject to treat a number of conditions including, for example, unregulated angiogenesis or blood vessel injury. The cells may also be stored in cryogenic conditions.

The progenitor cells are administered to a subject by any suitable means, including, for example, intravenous infusion, bolus injection, and site directed delivery via a catheter. Preferably, the progenitor cells obtained from the subject are readministered. Generally, from about 10⁶ to about 10¹⁸ progenitor cells are administered to a subject for transplantation. Depending on the use of the progenitor cells, various genetic material may be delivered to the cell (e.g., a polynucleotide encoding p75/TNFR2). Such genetic material includes nucleic acid sequences both exogenous and endogenous to cells into which a virus vector, for example, a pox virus such as swine pox containing the human p75/TNFR2 gene may be introduced. Additionally, it is of interest to use genes encoding polypeptides for secretion from the endothelial progenitor cells so as to provide for a systemic effect by the protein encoded by the gene. Specific genes of interest include those encoding p75/TNFR2, TGF-α, TGF-β, hemoglobin, interleukin-1, interleukin-2, interleukin-3, interleukin-4, interleukin-5, interleukin-6, interleukin-7, interleukin-8, interleukin-9, interleukin-10, interleukin-11, interleukin-12 etc., GM-CSF, G-CSF, M-CSF, human growth factor, co-stimulatory factor B7, insulin, factor VIII, factor IX, PDGF, EGF, NGF, IL-ira, EPO, β-globin, endothelial cell mitogens, as well as biologically active variants of these proteins. The gene may further encode a product that regulates expression of another gene product or blocks one or more steps in a biological pathway. Similarly, the gene may encode a therapeutic protein fused to a targeting polypeptide, to deliver a therapeutic effect to a diseased tissue or organ. To further enhance angiogenesis, endothelial cell mitogens may also be administered to the subject in conjunction with, or subsequent to, the administration of the endothelial progenitor cells. Endothelial cell mitogens can be administered directly, e.g., intra-arterially, intramuscularly, or intravenously, or a nucleic acid molecule encoding the mitogen may be used. See, Baffour, et al., J Vasc Surg. 16(2):181-91 (1992). (bFGF); Pu, et al, Circulation, 88:208-215 (1993) (aFGF); Yanagisawa-Miwa, et al., Science. 257(5075):1401-3 (1992). (bFGF); Ferrara, et al., Biochem. Biophys. Res. Commun., 161:851-855 (1989) (VEGF); (Takeshita, et al., Circulation, 90:228-234 (1994)).

The nucleic acid encoding the endothelial cell mitogen can be administered to a blood vessel by perfusing the ischemic tissue or to a site of vascular injury via a catheter, for example, a hydrogel catheter, as described by U.S. Ser. No. 08/675,523, the disclosure of which is herein incorporated by reference. The nucleic acid also can be delivered by injection directly into the ischemic tissue using the method described in U.S. Ser. No. 08/545,998.

As used herein the term “endothelial cell mitogen” means any protein, polypeptide, variant or portion thereof that is capable of, directly or indirectly, inducing endothelial cell growth. Such proteins include, for example, acidic and basic fibroblast growth factors (aFGF) (GenBank Accession No. NP_{—}149127) and bFGF (GenBank Accession No. AAA52448), vascular endothelial growth factor (VEGF) (GenBank Accession No. AAA35789 or NP_{—}001020539), epidermal growth factor (EGF) (GenBank Accession No. NP_{—}001954), transforming growth factor α (TGF-α) (GenBank Accession No. NP_{—}003227) and transforming growth factor β (TFG-β) (GenBank Accession No. 1109243A), platelet-derived endothelial cell growth factor (PD-ECGF) (GenBank Accession No. NP_{—}001944), platelet-derived growth factor (PDGF) (GenBank Accession No. 1109245A), hepatocyte growth factor (HGF) (GenBank Accession No. BAA14348), insulin like growth factor (IGF) (GenBank Accession No. P08833), erythropoietin (GenBank Accession No. P01588), colony stimulating factor (CSF), macrophage-CSF (M-CSF) (GenBank Accession No. AAB59527), granulocyte/macrophage CSF (GM-CSF) (GenBank Accession No. NP_{—}000749), monocyte chemotactic protein-1 (GenBank Accession No. P13500) and nitric oxide synthase (NOS) (GenBank Accession No. AAA36365). See, Klagsbrun, et al., Annu. Rev. Physiol., 53:217-239 (1991); Folkman, et al., J. Biol. Chem., 267:10931-10934 (1992) and Symes, et al., Current Opinion in Lipidology, 5:305-312 (1994). Variants or fragments of a mitogen may be used as long as they reduce inflammation, or induce or promote endothelial cell or endothelial progenitor cell growth. Preferably, the endothelial cell mitogen contains a secretory signal sequence that facilitates secretion of the protein. Proteins having native signal sequences, e.g., VEGF, are preferred. Proteins that do not have native signal sequences, e.g., bFGF, can be modified to contain such sequences using routine genetic manipulation techniques. See, Nabel et al., Nature, 362:844 (1993).

The nucleotide sequence of numerous endothelial cell mitogens, are readily available through a number of computer data bases, for example, GenBank, EMBL and Swiss-Prot. Using this information, a DNA segment encoding the desired may be chemically synthesized or, alternatively, such a DNA segment may be obtained using routine procedures in the art, e.g, PCR amplification. A DNA encoding VEGF is disclosed in U.S. Pat. No. 5,332,671, the disclosure of which is herein incorporated by reference.

In certain situations, it may be desirable to use nucleic acids encoding two or more different proteins in order to optimize the therapeutic outcome. For example, DNA encoding two proteins, e.g., VEGF and bFGF, can be used, and provides an improvement over the use of bFGF alone, or an angiogenic factor (e.g., fibroblast growth factor (bFGF), acidic FGF (aFGF), FGF-5, vascular endothelial growth factor isoforms (VEGF), angiopoietin-1 (Ang-1), angiopoietin-2 (Ang-2), platelet-derived endothelial cell growth factor (PD-ECGF), hepatocyte growth factor (HGF) (GenBank Accession No. BAA 14348), interleukin-8 (IL-8) (GenBank Accession No. NP_{—}000575), granulocyte-colony stimulating factor (G-CSF), placental growth factor (GenBank Accession No. NP_{—}002623.), proliferin (GenBank Accession No. S48671), angiogenin (NP_{—}001136), TNFα, Transforming growth factor-β (GenBank Accession No. 1109243A)) can be combined with other genes or their encoded gene products to enhance the activity of targeted cells, while simultaneously inducing angiogenesis, including, for example, nitric oxide synthase, L-arginine, fibronectin, urokinase, plasminogen activator and heparin.

The effective dose of the nucleic acid will be a function of the particular expressed protein, the target tissue, the subject and his or her clinical condition. Effective amount of DNA are between about 1 and 4000 μg, more preferably about 1000 and 2000, most preferably between about 2000 and 4000.

### Kits

The invention provides kits for the treatment or prevention of inflammation in a tissue or organ and for the repair or regeneration of tissue damage related to ischemia. In one embodiment, the kit includes a therapeutic or prophylactic composition containing an effective amount of a p75 receptor/TNFR2 polypeptide or an expression vector encoding the p75 receptor/TNFR2 polypeptide in unit dosage form. In some embodiments, the kit comprises a sterile container which contains a therapeutic or prophylactic composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired an expression vector of the invention is provided together with instructions for administering it to a subject having or at risk of developing ischemia. The instructions will generally include information about the use of the composition for the treatment or prevention of ischemia or for enhancing angiogenesis to a tissue in need thereof. In other embodiments, the instructions include at least one of the following: description of the expression vector; dosage schedule and administration for treatment or prevention of ischemia or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

### Formulation of Pharmaceutical Compositions

The administration of a compound for the treatment of inflammation or for the repair of tissue damage, i.e., damage related to ischemia, or the promotion of tissue regeneration may be by any suitable means that results in a concentration of the therapeutic that, combined with other components, is effective in preventing, ameliorating, reducing, or stabilizing an ischemic disease. The compound may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions according to the invention may be formulated to release the active compound substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in the central nervous system or cerebrospinal fluid; (v) formulations that allow for convenient dosing, such that doses are administered, for example, once every one or two weeks; and (vi) formulations that target inflammation by using carriers or chemical derivatives to deliver the therapeutic agent to a particular cell type (e.g., cell subject to reduced blood flow related to ischemia) whose function is disrupted by inflammation or ischemia. For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

Human dosage amounts for any therapy described herein can initially be determined by extrapolating from the amount of compound used in mice, as a skilled artisan recognizes it is routine in the art to modify the dosage for humans compared to animal models. In certain embodiments it is envisioned that the dosage may vary from between about 1 mg compound/Kg body weight to about 5000 mg compound/Kg body weight; or from about 5 mg/Kg body weight to about 4000 mg/Kg body weight or from about 10 mg/Kg body weight to about 3000 mg/Kg body weight; or from about 50 mg/Kg body weight to about 2000 mg/Kg body weight; or from about 100 mg/Kg body weight to about 1000 mg/Kg body weight; or from about 150 mg/Kg body weight to about 500 mg/Kg body weight. In other embodiments this dose may be about 1, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, 5000 mg/Kg body weight. In other embodiments, it is envisaged that higher does may be used, such doses may be in the range of about 5 mg compound/Kg body to about 20 mg compound/Kg body. In other embodiments the doses may be about 8, 10, 12, 14, 16 or 18 mg/Kg body weight. Of course, a dosage amount may be adjusted upward or downward, as is routinely done in such treatment protocols, depending on the results of the initial clinical trials and the needs of a particular patient.

### Screening Assays

Compositions of the invention are useful for the high-throughput low-cost screening of candidate compounds that enhance p75/TNFR2 expression or activity. Such compounds are useful for the treatment or prevention of inflammation or for promoting the activation, survival, or proliferation in a damaged tissue. Tissues or cells treated with a candidate compound are compared to untreated control samples to identify therapeutic agents that enhance the p75 receptor/TNFR2 expression or activity. If desired, such compounds are further tested in vitro or in vivo for their effects on angiogenesis using any method known in the art. Any number of methods are available for carrying out screening assays to identify new candidate compounds that bind a p75 receptor/TNFR2 polypeptide and reduce inflammation or stem cell activation.

In one working example, candidate compounds are added at varying concentrations to the culture medium of cultured cells. p75 receptor/TNFR2 expression (e.g., polypeptide or mRNA expression) is then measured using standard methods. The expression of a p75 receptor/TNFR2 in the presence of the candidate compound is compared to the level measured in a control culture medium lacking the candidate molecule. A compound that increases the expression of a p75 receptor/TNFR2 is useful for promoting an increase in angiogenesis. Such compounds are considered useful in the invention; such a compound may be used, for example, as a therapeutic to prevent, delay, ameliorate, stabilize, reduce the severity of, or treat ischemia. In other embodiments, the candidate compound prevents, delays, ameliorates, stabilizes, or treats a disease or disorder related to inflammation, ischemia, tissue damage, or apoptosis associated with ischemia. Such therapeutic compounds are useful in vivo as well as ex vivo.

In some embodiments, a compound that promotes an increase in the biological activity of a p75 receptor/TNFR2 of the invention is considered useful. Such compounds are added to a culture containing p75 receptor/TNFR2 expressing cells. The effect of the compound on p75 receptor/TNFR2 biological activity is measured and compared to p75 receptor/TNFR2 biological activity in the absence of the candidate compound. Again, a candidate compound that enhances the biological activity of a p75 receptor/TNFR2 may be used, for example, as a therapeutic to treat or prevent ischemia.

One skilled in the art appreciates that the effects of a candidate compound on the p75 receptor/TNFR2 expression or biological activity are typically compared to the expression or activity of the p75 receptor/TNFR2 in the absence of the candidate compound. Thus, the screening methods include comparing the value of a cell modulated by a candidate compound to a reference value of an untreated control cell.

Expression levels can be compared by procedures well known in the art such as RT-PCR, Northern blotting, Western blotting, flow cytometry, immunocytochemistry, binding to magnetic and/or antibody-coated beads, in situ hybridization, fluorescence in situ hybridization (FISH), flow chamber adhesion assay, and ELISA, microarray analysis, or colorimetric assays, such as the Bradford Assay and Lowry Assay. Changes in angiogenesis can be assayed by methods described herein or by any method known in the art, including Angiogram, Computed Tomography Angiography (CTA), Duplex Ultrasound, magnetic resonance angiography, vascular ultrasound, or angiogram.

Molecules that increase the p75 receptor/TNFR2 expression or activity include organic molecules, peptides, peptide mimetics, polypeptides, nucleic acids, and antibodies that bind to a the p75 receptor/TNFR2 nucleic acid sequence or polypeptide and increase its expression or biological activity are preferred.

In yet another example, candidate compounds are screened for those that specifically bind to a p75 receptor/TNFR2. The efficacy of such a candidate compound is dependent upon its ability to interact with the p75 receptor/TNFR2, or with functional equivalents thereof. Such an interaction can be readily assayed using any number of standard binding techniques and functional assays (e.g., those described in Ausubel et al., supra). In one embodiment, the compound is assayed in vitro for receptor binding.

In one particular working example, a candidate compound that binds to a p75 receptor/TNFR2 is identified using a chromatography-based technique. For example, a recombinant polypeptide of the invention may be purified by standard techniques from cells engineered to express the polypeptide (e.g., those described above) and may be immobilized on a column. A solution of candidate compounds is then passed through the column, and a compound specific for the p75 receptor/TNFR2 is identified on the basis of its ability to bind to the polypeptide and be immobilized on the column. To isolate the compound, the column is washed to remove non-specifically bound molecules, and the compound of interest is then released from the column and collected. Similar methods may be used to isolate a compound bound to a polypeptide microarray. Compounds identified using such methods are then assayed for their effect on angiogenesis as described herein.

In another example, the compound, e.g., the substrate, is coupled to a radioisotope or enzymatic label such that binding of the compound to the p75 receptor/TNFR2 can be determined by detecting the labeled compound, e.g., substrate, in a complex. For example, compounds can be labeled with ¹²⁵I, ³⁵S, ¹⁴C, or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

In yet another embodiment, a cell-free assay is provided in which the p75 receptor/TNFR2 or a biologically active portion thereof is contacted with a test compound and the ability of the test compound to bind to the polypeptide thereof is evaluated.

The interaction between two molecules can also be detected, e.g., using fluorescence energy transfer (FET) (see, for example, Lakowicz et al., U.S. Pat. No. 5,631,169; Stavrianopoulos et al., U.S. Pat. No. 4,868,103). A fluorophore label on the first, ‘donor’ molecule is selected such that its emitted fluorescent energy will be absorbed by a fluorescent label on a second, ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determining the ability of a test compound to bind to the p75 receptor/TNFR2 can be accomplished using real-time Biomolecular Interaction Analysis (BIA) (see, e.g., Sjolander, S., and Urbaniczky, C., Anal. Chem. 63:2338-2345, 1991; and Szabo et al., Curr. Opin. Struct. Biol. 5:699-705, 1995). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal that can be used as an indication of real-time reactions between biological molecules.

It may be desirable to immobilize either the candidate compound or its p75 receptor/TNFR2 target to facilitate separation of complexed from uncomplexed forms of one or both of the proteins, as well as to accommodate automation of the assay. Binding of a candidate compound to the p75 receptor/TNFR2, or interaction of a test compound with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/p75 receptor/TNFR2 fusion proteins can be adsorbed onto glutathione sepharose beads (Sigma Chemical, St. Louis, Mo.) or glutathione derivatized microtiter plates, which are then combined with the test compound or the test compound and a sample comprising the GST-tagged p75 receptor/TNFR2 polypeptide, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Other techniques for immobilizing a complex of a compound and the p75 receptor/TNFR2 polypeptide on matrices include using conjugation of biotin and streptavidin. For example, biotinylated proteins can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-Ig antibody).

In one embodiment, a p75 receptor/TNFR2 antibody is identified that reacts with an epitope on the p75 receptor/TNFR2. Methods for detecting binding of expression antibody to the receptor are known in the art and include immunodetection of complexes, as well as enzyme-linked assays which rely on detecting an enzymatic activity associated with the channel. Antibodies that bind the p75 receptor/TNFR2 are then tested for the ability to activate the receptor. Such antibodies may test for angiogenesis promoting activity as described herein.

Alternatively, cell free assays can be conducted that assay the interaction of a compound with a p75 receptor/TNFR2 or that assay the activity of a p75 receptor/TNFR2. In such an assay, the reaction products are separated from unreacted components, by any of a number of standard techniques, including but not limited to: differential centrifugation (see, for example, Rivas, G., and Minton, A. P., Trends Biochem Sci 18:284-7, 1993); chromatography (gel filtration chromatography, ion-exchange chromatography); electrophoresis and immunoprecipitation (see, for example, Ausubel, F. et al., eds. (1999) Current Protocols in Molecular Biology, J. Wiley: New York). Such resins and chromatographic techniques are known to one skilled in the art (see, e.g., Heegaard, N. H., J Mol Recognit 11:141-8, 1998; Hage, D. S., and Tweed, S. A., J Chromatogr B Biomed Sci Appl. 699:499-525, 1997). Further, fluorescence energy transfer may also be conveniently utilized, as described herein, to detect binding without further purification of the complex from solution. Preferably, cell free assays preserve the structure of the p75 receptor/TNFR2, e.g., by including a membrane component or synthetic membrane components.

In a specific embodiment, the assay includes contacting the p75 receptor/TNFR2 polypeptide or biologically active portion thereof with a known compound which binds the p75 receptor/TNFR2 polypeptide to form an assay mixture, contacting the assay mixture with a test compound, and determining the ability of the test compound to interact with a the p75 receptor/TNFR2, wherein determining the ability of the test compound to interact with a the p75 receptor/TNFR2 includes determining the ability of the test compound to preferentially bind to the p75 receptor/TNFR2, or to modulate the activity of the p75 receptor/TNFR2, as compared to the known compound.

Compounds isolated by this method (or any other appropriate method) may, if desired, be further purified (e.g., by high performance liquid chromatography). In addition, these candidate compounds may be tested for their ability to increase the activity of a p75 receptor/TNFR2 (e.g., as described herein). Compounds isolated by this approach may also be used, for example, as therapeutics to treat or prevent ischemia in a subject. Compounds that are identified as binding to the p75 receptor/TNFR2 with an affinity constant less than or equal to 10 mM are considered particularly useful in the invention. Alternatively, any in vivo protein interaction detection system, for example, any two-hybrid assay may be utilized.

In another embodiment, a the p75 receptor/TNFR2 nucleic acid described herein is expressed as a transcriptional or translational fusion with a detectable reporter, and expressed in an isolated cell (e.g., mammalian or insect cell) under the control of an endogenous or a heterologous promoter. The cell expressing the fusion protein is then contacted with a candidate compound, and the expression of the detectable reporter in that cell is compared to the expression of the detectable reporter in an untreated control cell. A candidate compound that increases the expression of the detectable reporter is a compound that is useful for the treatment of ischemia. In preferred embodiments, the candidate compound increases the expression of a reporter gene fused to the p75 receptor/TNFR2 nucleic acid molecule.

Each of the DNA sequences listed herein may also be used in the discovery and development of a therapeutic compound for the treatment of inflammation. The encoded protein, upon expression, can be used as a target for the screening of drugs. Additionally, the DNA sequences encoding the amino terminal regions of the encoded protein or Shine-Delgarno or other translation facilitating sequences of the respective mRNA can be used to construct sequences that promote the expression of the coding sequence of interest. Such sequences may be isolated by standard techniques (Ausubel et al., supra).

Small molecules of the invention preferably have a molecular weight below 2,000 daltons, more preferably between 300 and 1,000 daltons, and most preferably between 400 and 700 daltons. It is preferred that these small molecules are organic molecules.

### Test Compounds and Extracts

In general, compounds capable of increasing the expression or activity of the p75 receptor/TNFR2 are identified from large libraries of both natural product or synthetic (or semi-synthetic) extracts or chemical libraries or from polypeptide or nucleic acid libraries, according to methods known in the art. Those skilled in the field of drug discovery and development will understand that the precise source of test extracts or compounds is not critical to the screening procedure(s) of the invention. Compounds used in screens may include known compounds (for example, known therapeutics used for other diseases or disorders). Alternatively, virtually any number of unknown chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds.

Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, chemical compounds to be used as candidate compounds can be synthesized from readily available starting materials using standard synthetic techniques and methodologies known to those of ordinary skill in the art. Synthetic chemistry transformations and protecting group methodologies (protection and deprotection) useful in synthesizing the compounds identified by the methods described herein are known in the art and include, for example, those such as described in R. Larock, Comprehensive Organic Transformations, VCH Publishers (1989); T. W. Greene and P. G. M. Wuts, Protective Groups in Organic Synthesis, 2nd ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Examples of methods for the synthesis of molecular libraries can be found in the art, for example in: DeWitt et al., Proc. Natl. Acad. Sci. U.S.A. 90:6909, 1993; Erb et al., Proc. Natl. Acad. Sci. USA 91:11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl. 33:2061, 1994; and Gallop et al., J. Med. Chem. 37:1233, 1994. Furthermore, if desired, any library or compound is readily modified using standard chemical, physical, or biochemical methods.

Libraries of compounds may be presented in solution (e.g., Houghten, Biotechniques 13:412-421, 1992), or on beads (Lam, Nature 354:82-84, 1991), chips (Fodor, Nature 364:555-556, 1993), bacteria (Ladner, U.S. Pat. No. 5,223,409), spores (Ladner U.S. Pat. No. 5,223,409), plasmids (Cull et al., Proc Natl Acad Sci USA 89:1865-1869, 1992) or on phage (Scott and Smith, Science 249:386-390, 1990; Devlin, Science 249:404-406, 1990; Cwirla et al. Proc. Natl. Acad. Sci. 87:6378-6382, 1990; Felici, J. Mol. Biol. 222:301-310, 1991; Ladner supra.).

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity should be employed whenever possible.

When a crude extract is found to increase the activity of a p75 receptor/TNFR2, or to bind the p75 receptor/TNFR2, further fractionation of the positive lead extract is necessary to isolate chemical constituents responsible for the observed effect. Thus, the goal of the extraction, fractionation, and purification process is the careful characterization and identification of a chemical entity within the crude extract that increases the activity of a the p75 receptor/TNFR2. Methods of fractionation and purification of such heterogenous extracts are known in the art. If desired, compounds shown to be useful as therapeutics for the treatment of ischemia are chemically modified according to methods known in the art.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### Examples

Neovascularization was studied in a mouse hind limb ischemia (HLI) model in young and old p75 TNFR2 knockout (p75KO) and wild type (WT) age-matched controls. In this model, the femoral artery of one hindlimb was ligated and excised to induce hindlimb ischemia. Murine models of hindlimb ischemia are known in the art, and are described, for example, by Couffinhal et al., American Journal of Pathology, Vol 152, 1667-1679, 1998. Between days 7 and 10 post-hindlimb surgery 100% of old p75KOs experienced auto-amputation of the operated limbs, whereas none of the age-matched WT mice exhibited hindlimb necrosis. Mild to moderate necrosis of the distal ischemic hindlimb was observed in 100% of young p75KO mice beginning at day 7-14, but not evidence of hindlimb necrosis in the young wild-type mice. Poor blood flow recovery in p75KO mice was associated with decreased capillary density and significant reduction in the expression of VEGF and bFGF2 mRNA transcripts in ischemic tissue and in circulating endothelial progenitor cells (EPCs). Moreover, the number of circulating bone marrow-derived endothelial progenitor cells was significantly reduced in p75 KO mice. Compared to presurgery, on days 1-10 post-hindlimb surgery there was 6-10-fold increase in the number of sattelite-cells (as shown by embryonic NCAM staining) in wild-type mice, whereas in p75KOs after day 1 and through day 10 satellite cells were not detectable. To the contrary, p75KO tissue showed increased and prolonged (up to day 10) inflammation—neutrophil (MPO-1) and macrophage (F/480) infiltration. Transplantation of wild-type bone marrow mononuclear cells (MNC) into gamma-irradiated p75KO mice one month prior to hindlimb surgery prevented limb loss and preserved limb muscle mass, suggesting that ischemia-induced neovascularization is mediated, at least in part, via p75 TNF receptor expressed in BM derived cells.

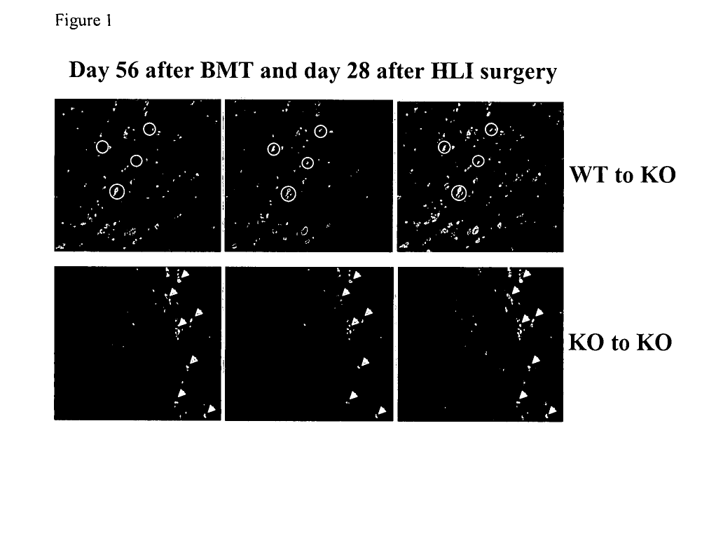

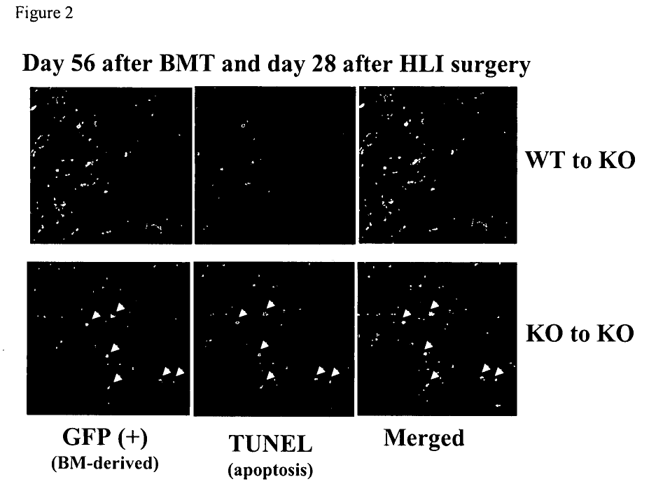

In the same BM transplantation model, the rate of proliferation (Ki67+ cells) of resident GFP (−) vs BM-derived GFP (+) cells was evaluated. In both WT and p75KO ischemic tissue Ki67 (+) cells almost exclusively were GFP (+), indicating that only BM-derived cells proliferate in the ischemic tissue. Interestingly, Ki67/GFP (+) cells started to appear in WT tissue by day 3 through day 21, whereas in p75KO tissue first proliferative activity was detected on day 28, suggesting extremely delayed recovery and regenaration in p75KO tissue. FIG. 1 shows that bone marrow-derived wild-type (WT) and TNFR2/p75 knockout (p75KO) cells from young mice proliferated in old p75KO ischemic tissue. Significantly, resident cells proliferated only in old p75KO mice that were transplanted with young wild-type marrow. FIG. 2 shows that bone marrow-derived WT cells from young mice survive in old p75KO ischemic tissue. No cells from young bone marrow-derived p75KO cells survived in old p75KO ischemic tissue.

These studies indicated that signaling through p75 receptor is required for collateral vessel development in ischemia-induced neovascularization and also plays a role in muscle preservation or regeneration. Furthermore, these results indicate that the p75 receptor modulation could be used to improve the repair of ischemic tissue in adult vascular diseases.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method of treating or preventing inflammation or repairing or regenerating a damaged tissue in a subject, the method comprising,
a) contacting a cell of the subject with a p75/TNFR2 nucleic acid molecule encoding a p75/TNFR2 polypeptide or a fragment thereof; and
b) expressing the p75/TNFR2 polypeptide in the cell, wherein the method treats or prevents inflammation in the subject.

2. (canceled)

3. The method of claim 1, wherein the tissue damage is associated with ischemia.

4. A method for repairing or regenerating a damaged tissue, the method comprising,
a) contacting a cell of the subject with a nucleic acid molecule encoding a p75/TNFR2 polypeptide or a fragment thereof;
b) expressing the p75/TNFR2 polypeptide in the cell, thereby repairing or regenerating the damaged tissue.

5. The method of claim 1, wherein the cell is a skeletal muscle cell, endothelial cell, resident stem cell, satellite cell, cardiomyocyte, or cardiac stem cell.

6. A method of treating tissue damage in a subject comprising:
a) contacting an adult stem isolated from the subject with a nucleic acid molecule encoding a p75/TNFR2 polypeptide or a fragment thereof; and
b) administering the cell to the subject, wherein the method enhances angiogenesis.

7. (canceled)

8. A method for increasing the survival of a transplanted stem cell in an damaged tissue, the method comprising,
a) contacting a cell in a damaged tissue of the subject with a nucleic acid molecule encoding a p75/TNFR2 polypeptide or a fragment thereof;
b) expressing the p75/TNFR2 polypeptide in the cell, and
c) transplanting the cell into the damaged tissue, wherein the method increases the survival of the stem cell in the damaged tissue.

9. The method of claim 8, wherein the method enhances the local release of angiogenic growth factors and cytokines in the tissue.

10. The method of claim 8, further comprising the steps of administering to the subject an angiogenic factor selected from the group consisting of: vascular endothelial growth factor (VEGF), hepatocyte growth factor (HGF), basic fibroblast growth factor (bFGF), angiopoietin 1, angiopoietin 2 and monocyte chemotactic protein-1 (MCP-1).

11. The method of claim 8, further comprising the steps of administering to the subject an endothelial cell mitogen selected from the group consisting of acidic and basic fibroblast growth factors, vascular endothelial growth factor, epidermal growth factor, transforming growth factor α and β, platelet-derived endothelial growth factor, platelet-derived growth factor, hepatocyte growth factor, insulin like growth factor, erythropoietin, colony stimulating factor, macrophage-CSF, granulocyte/macrophage CSF and nitric oxide synthase.

12. The method of claim 1, wherein the cell is in vivo or in vitro.

13-14. (canceled)

15. The method of claim 14, wherein the cell is delivered systemically or directly to a damaged tissue.

16-20. (canceled)

21. The method of claim 6, wherein the tissue damage is related to ischemia.

22. The method of claim 1, wherein the method treats an inflammatory disease selected from the group consisting of arthritis, rheumatoid arthritis, Crohn's disease, ulcerative colitis, asthma, chronic obstructive pulmonary disease, polymylagia rheumatica, giant cell arteritis, systemic lupus erythematosus, atopic dermatitis, multiple sclerosis, myasthenia gravis, psoriasis, ankylosing spondylitis, and psoriatic arthritis.

23. The method of claim 1, wherein the method treats a condition selected from the group consisting of myocardial infarction, heart failure, congestive heart failure, a stroke, a transient ischemic episode, a reperfusion injury, physical injury, renal failure, a secondary exsanguination, stroke, and traumatic brain injury, a transient ischemic attack.

24-26. (canceled)

27. A kit for the treatment or prevention of inflammation, the kit comprising an effective amount of an expression vector encoding a human p75/TNFR2 polypeptide or a fragment thereof in a pharmaceutically acceptable excipient, wherein the p75/TNFR2 polypeptide is operably linked to a promoter sufficient to drive expression of the p75/TNFR2 polypeptide in a mammalian cell, and written instructions for the use of the kit.

28. A method of monitoring a subject being treated for inflammation, the method comprising:
a) administering a treatment that enhances the expression of a p75/TNFR2 polypeptide in a cell of the subject; and
b) measuring inflammation in a tissue of the subject relative to a reference, wherein an decrease in inflammation indicates a reduced severity of ischemia in the subject.

29-31. (canceled)

32. A method for identifying a candidate compound useful for the treatment of inflammation, the method comprising the steps of:
(a) contacting a cell expressing p75/TNFR2 nucleic acid molecule with a candidate compound; and
(b) detecting an increase in p75/TNFR2 expression in the cell relative to a reference, wherein an increase in p75/TNFR2 expression identifies the candidate compound as a compound useful for the treatment of ischemia.

33. The method of claim 32, wherein the method identifies a compound that increases transcription or translation of a p75/TNFR2 nucleic acid molecule or increases stabilization and decreases degradation of gene product of p75/TNFR2 nucleic acid molecule.

34-35. (canceled)

36. A method for identifying a candidate compound a candidate compound useful for the treatment of inflammation, the method comprising the steps of:
(a) contacting a cell expressing p75/TNFR2 polypeptide with a candidate compound; and
(b) detecting an increase in the level of p75/TNFR2 polypeptide in the cell relative to a reference level, wherein an increase in the level of p75/TNFR2 polypeptide identifies a candidate compound useful for the treatment of ischemia.

37. A method for identifying a candidate compound useful for the treatment of ischemia, the method comprising the steps of:
(a) contacting a cell expressing a p75/TNFR2 polypeptide with a candidate compound; and
(b) detecting an increase in the biological activity of the p75/TNFR2 polypeptide in the cell contacted with the candidate compound with a reference level of biological activity wherein the candidate compound as a candidate compound that useful for the treatment of ischemia.
